(19) United States
(12) Patent Application Publication
(10) Pub. No.: US 2023/0277856 A1
(43) Pub. Date: Sep. 7, 2023

(54) AN INTRALUMINAL CONTRACTION AUGMENTATION SYSTEM

(71) Applicant: National University of Ireland, Galway, Galway (IE)

(72) Inventors: Faisal SHARIF, Galway (IE); Shahen ANDONIAN, Galway (IE); Conor HURLEY, Galway (IE); Ben Nadav HAIM, Galway (IE)

(21) Appl. No.: 18/010,325

(22) PCT Filed: Jun. 15, 2021
(86) PCT No.: PCT/EP2021/066147

§ 371 Date: Dec. 14, 2022

(30) Foreign Application Priority Data
Jun. 16, 2020 (EP) 20180264.2

(51) Int. Cl. A61N 1/365; A61F 5/00

(52) U.S. Cl. CPC A61N 1/36514; A61F 5/0046

(57) ABSTRACT

The present invention provides a system for augmenting the contraction of a contractile organ in a subject. The system comprises at least one implantable organ contraction device comprising an electronic linear actuation device (1) for producing a contraction force; an anchoring assembly (13,14) for operably coupling the electronic linear actuation device to at least one wall of the contractile organ; and a controller (65) configured to modify the output parameters of the electronic linear actuation device so as to activate the electronic linear actuation device in a pattern synergistic to the natural contraction cycle of the contractile organ.

### FIELD OF THE INVENTION

The present invention relates to an intra-luminal contraction augmentation system. Also contemplated are methods of treating diseases related to reduced contraction of body lumens with said intraluminal contraction augmentation system. Diseases that may be treated by the contraction augmentation device include heart disease, intestinal motility disorders (e.g. gastroparesis, paralytic ileus) and incomplete urinary bladder emptying.

### BACKGROUND TO THE INVENTION

Several hollow contractile organs of the human body contract in order to increase pressure in the organ and propel material out of the organ. Contractile hollow organs include the heart, arteries, urinary bladder, ureters, uterus, gallbladder, bile ducts, oesophagus, stomach and intestines. These organs often suffer from reduced contraction capability, leading to several unmet medical needs and conditions that lead to reduced quality of life, increased infection, increased hospitalisation, increased morbidity and mortality. These conditions include heart failure, gastrointestinal motility disorders and bladder hypo-contractility disorders.

Heart failure (HF) is a complex clinical syndrome that results from any structural or functional impairment of ventricular filling or ejection of blood. The cardinal manifestations of HF are dyspnea and fatigue, which may limit exercise tolerance, and fluid retention, which may lead to pulmonary and/or splanchnic congestion and/or peripheral oedema.

HF with reduced ejection fraction (HFrEF) is commonly caused by ischemic heart disease. HF leads to decrease in stroke volume and cardiac output which results in the activation of neurohormonal response in order to restore the normal cardiac output. However, this neurohormonal response causes further stress of left ventricular (LV) wall and ventricular dilatation leading to worsening of ventricular function.

Despite improvements in the treatment of HFrEF, acute heart failure remains a substantial unmet clinical need with high morbidity and mortality rates. Patients with acute heart failure syndromes (AHFSs) can present with three pathophysiological forms: (a) acute pulmonary oedema, (b) cardiogenic Shock (5-8% of (ST Elevation Myocardial Infarction) STEMI and 2.5% of non-STEMI), and (c) acute decompensation of CHF. From 2008 to 2011, 24.8% of patients presented with the diagnosis of chronic heart failure received a permanent device with resultant survival benefit and improved quality of life. Patients with Left Ventricular Assist Device (LVAD) develop significant device malfunctions in 10% of patients. Bleeding, right heart failure, stroke, infection and device failure are among the main complications, with 55% chance of rehospitalisation for any cause, 30% chance of major bleeding within the first month, 20% chance of major bleeding after first month, 20% chance of serious device-related infection, 5% chance of device malfunction due to clotting, and 18% chance of ongoing heart failure. In addition, current treatment options, including LVAD placement and heart transplants, are considered extremely invasive procedures.

There is a need to reduce morbidity and mortality in particular with respect to acute cardiogenic shock to improve the prognosis and the patients' quality of life. For this purpose, there is a need to develop individualized programable intra-ventricular implants that will improve patient outcomes.

Gastrointestinal motility disorders are caused by disfunction of nerves and muscles of the gastrointestinal tract leading to reduced muscle contraction. This may cause lumen obstructions anywhere throughout the gastrointestinal tract and may impair digestion. Gastrointestinal motility disorders are mostly chronic disorders and cause a substantial decrease in the quality of life of patients. They include gastroparesis and paralytic ileus. Gastroparesis is the chronic paralysis of the stomach muscles. Patients with gastroparesis suffer from impaired emptying of the stomach resulting from a decrease in the contractions that propel food out of the stomach. Patients experience interference in normal digestion. Symptoms include heartburn or GERD, vomiting, abdominal bloating, nausea, impaired blood sugar control and weight loss. Gastroparesis may be caused by damage to the vagus nerve which may result from uncontrolled diabetes, gastric surgery or certain medications (e.g. antidepressants). Gastroparesis is a chronic condition with no cure. The condition severely decreases quality of life, can cause significant complications, and may reduce survival. Paralytic ileus is caused by complete or partial paralysis of the intestinal muscles leading to functional obstruction of the intestine. The condition may be temporary, but if left untreated, it can lead to the death of intestinal tissue and severe infections of the abdominal cavity. There is a need for a device that increases contraction of the gastrointestinal tract to improve the emptying of its contents and prevent gastrointestinal obstructions.

Overflow incontinence, also known as underactive bladder, may result from detrusor underactivity (DUA). The detrusor muscle is smooth muscle of the bladder wall. Its function is to contract during urination to eject urine from the bladder to the urethra. DUA may be caused by damage to the nerves innervating the bladder or damage to the detrusor muscle. Loss of detrusor muscle contractility leads to incomplete emptying of the bladder and may result in urinary infection and kidney damage.

It is an object of the present invention to provide a device to treat overflow incontinence caused by DUA.

WO2017223508 (Harvard) discloses a ventricular assist device that may be employed as an assist device for the left or right ventricle. The device comprises a septal anchor, a brace that abuts a free external wall of the ventricle, and a shaft connecting the anchor and brace. Actuators are associated with the brace that are actuated to expand towards the anchor compressing the ventricle. The actuator of this device is pneumatically operated, and is thus required to be connected to an external pump that is required to be carried by the patient at all times. In addition, highly invasive surgery is required to implant the device, while the pneumatic actuation system is at risk of puncture during implantation.

WO2015112971 discloses an implantable device for providing contractile assistance to a heart. US2006178550 discloses a ventricular assist device to provide cardiac assistance to a damaged ventricle chamber.

It is an object of the invention to overcome at least one of the above-referenced problems.

### SUMMARY OF THE INVENTION

The present invention provides a computer implemented system, as set out in the appended claims, for augmenting the contraction of a contractile organ in a subject. The system comprises:
at least one implantable organ contraction device comprising an electronic linear actuation device for producing a contraction force;
an anchoring assembly for operably coupling the electronic linear actuation device to at least one wall of the contractile organ;
and
a controller configured to modify the output parameters of the electronic linear actuation device so as to activate the electronic linear actuation device in a pattern synergistic to the natural contraction cycle of the contractile organ.

In one embodiment, the system further comprises electronic circuitry for setting the output parameters of the electronic linear actuation device, wherein the electronic circuitry is coupled between the controller and the electronic linear actuation device and a power unit associated with the electronic circuitry, wherein the controller is configured to send control signals to the electronic circuitry to modify the output parameters of the electronic linear actuation device.

In one embodiment, the power unit comprises an implantable battery.

In one embodiment, the electronic circuitry comprises one or more of: a voltage multiplier, a duty cycle modifier, a current multiplier, a duration of output modifier and a frequency modifier.

In one embodiment, the anchoring assembly comprises at least one anchor configured to be deployed and embedded in tissue of the wall of the contractile organ.

In one embodiment, the system further comprises:
at least one sensor in communication with the controller for detecting one or more parameters associated with the contractile organ;
wherein the controller is configured to modify the output parameters of the electronic linear actuation device based on the one or more detected parameters.

In one embodiment, the contractile organ comprises the heart and wherein the at least one sensor comprises one of: a ventricle pressure sensor, a wireless pressure ventricular pressure sensor, a mems pressure sensor, an artery pressure sensor, a cardiac output (CO) sensor, a blood pressure sensor, a heart rate sensor, a motion sensor, an accelerometer, an ECG (Electrocardiogram) sensor, an O₂ saturation sensor, a microaccelerometer, a sonomicrometer, and a real-time contractility sensor.

In one embodiment, the anchoring assembly comprises a first anchor adapted to engage a septum of the heart ventricle and a second anchor adapted to engage a free wall of the heart ventricle, and wherein the electronic linear actuation device is configured to activate to extend and contract pulling the septum and free wall of the heart ventricle towards and away from each other repeatedly.

In one embodiment, the contractile organ comprises the stomach and wherein the at least one sensor comprises one of: a sensor for detecting electrical activity of the stomach, a sensor for detecting muscle contractility and a sensor for detecting chyme in the stomach beyond a specific volume.

In one embodiment, the controller is configured to deactivate the electronic linear actuation device upon the at least one sensor detecting the volume of chyme in the stomach to be below a predetermined threshold.

In one embodiment, the anchoring assembly comprises a first anchor adapted to engage one wall of the stomach and a second anchor adapted to engage another wall of the stomach, and wherein the electronic linear actuation device is configured to activate to expand and contract to move the walls of the stomach towards and away from each other.

In one embodiment, the contractile organ comprises the urinary bladder and the system further comprises:
a user activatable external control unit in communication with the controller;
wherein the controller is configured to modify the output parameters of the electronic linear actuation device upon activation of the external control unit by the subject.

In one embodiment, the system further comprises:
at least one sensor in communication with the controller for detecting one or more parameters associated with the bladder;
wherein the at least one sensor is configured to alert the subject to activate the external control unit upon detection of a full bladder.

In one embodiment, the controller is configured to deactivate the electronic linear actuation device upon the at least one sensor detecting that the intra-bladder pressure has dropped below a predetermined threshold value.

In one embodiment, the at least one sensor comprises a sensor for measuring intravesical bladder pressure.

In one embodiment, the anchoring assembly comprises a first anchor adapted to engage one wall of the bladder and a second anchor adapted to engage another wall of the bladder, and wherein the electronic linear actuation device is configured to activate to expand and contract to move the walls of the bladder towards and away from each other.

In one embodiment, the electronic linear actuation device comprises at least one electromagnetic linear actuation device.

In one embodiment, the electromagnetic linear actuation device comprises a first and a second electromagnetic actuator configured in one of: the first actuator back-to-back with the second actuator, the first actuator on top of the second actuator, the first actuator parallel to the second actuator or the first actuator colinear to the second actuator.

In one embodiment, the at least one of the first and the second electromagnetic actuators is configured to effect rotational actuation.

In one embodiment, the at least part of the electronic linear actuation device is disposed within a fluid impermeable biocompatible sleeve.

In one embodiment, the anchoring assembly comprises two or more anchors, and wherein the coupling between at least one of the anchors and the electronic linear actuation device comprises a magnet or magnetisable element.

In one embodiment, the anchoring assembly is encapsulated with an elastic polymer membrane that expands circumferentially when the anchoring assembly is compressed at deployment configuration.

In one embodiment, the system further comprises a memory in communication with the controller, wherein the controller is configured to modify the output parameters of the electronic linear actuation device in accordance with program instructions in the memory.

In one embodiment, the output parameters comprise one or more of: actuation frequency, actuation force, actuation acceleration rate and travel distance of actuation armature of the contraction device.

In an embodiment of the invention, the at least one electronic linear actuation device for producing a contraction force comprises an electromagnetic linear actuator.

In one embodiment, the electronic linear actuation device comprises one electromagnetic actuator.

In another embodiment the electronic linear actuation device comprises two electromagnetic actuators.

In yet another embodiment the electronic linear actuation device comprises two electromagnetic actuators positioned co-linearly to each other.

In a further embodiment, the electronic linear actuation device comprises two back-to-back electromagnetic actuators.

In a further embodiment, two or more electronic linear actuators are connected via a tethering means.

In yet a further embodiment the electronic linear actuation device comprises two electromagnetic actuators positioned one on top of the other.

In yet another embodiment the electronic linear actuation device comprises two electromagnetic actuators positioned parallel to each other.

In yet another embodiment the electronic linear actuation device comprises two electromagnetic actuators positioned at 0°≥Θ≤90° degrees to each other.

In an additional embodiment, two or more electromagnetic actuators are connected (at the opposite ends to the armatures and anchors) to a node that allows a variety of angles between the central axes of the various linear actuators.

In an embodiment of the invention, the electronic linear actuation device for producing a contraction force comprises a moving-iron (solenoid) electromagnetic linear actuator.

In a further embodiment of the invention, the at least one electronic linear actuation device for producing a contraction force comprises a moving-coil electromagnetic linear actuator.

In yet a further embodiment of the invention, the at least one electronic linear actuation device for producing a contraction force comprises a moving-magnet electromagnetic linear actuator.

In one embodiment, at least part, and preferably all, of the linear actuator is disposed within a fluid impermeable biocompatible sleeve. In one embodiment, the sleeve is compliant and configured to stretch during expansion of the actuator. In this embodiment, the compliant sleeve is in a stretched state when the armatures are fully extended, providing additional potential contraction energy during the contraction phase of the linear actuator.

In one embodiment, the system harnesses the expanding force of the organ through an elastic sleeve that encapsulates the linear actuation device.

In another embodiment, the system harnesses the expanding force of the organ through a spring arrangement.

In one embodiment, the sleeve is a non-compliant foldable structure configured to unfold to accommodate expansion of the actuator.

In one embodiment, the sleeve is a silicone capsule.

In one embodiment, one or more distal ends of one or more armatures are typically exposed proud of the end of the sleeve.

In one embodiment, power supply to the linear actuator is provided via the armature ends and through the anchoring arrangement. In this embodiment, it is not necessary to have an incision/hole separate to the anchoring holes in the organ (or chamber) wall specifically for power supply to the intraluminal linear actuator.

In one embodiment, power supply to the linear actuator is provided via a separate incision or hole in the organ (or chamber) wall specifically for power supply to the intraluminal linear actuator.

In an embodiment of the invention, a power unit such as a battery is embedded in the system. This battery can be charged wirelessly using electromagnetic inductive charging by means of a charging pad. Charging energy is sent through an inductive coupling to the battery. The charging pad has optimum fast rate wireless charging. The charging pad power cable is plugged into a wall outlet and the pad is placed inside a specially designed elastic chest belt with a pocket to place and hold the pad over the chest adjacent to the left lower rib cage for the period of charging.

In one embodiment the anchoring element is made of bare metal.

In one embodiment, the anchoring element comprises a shape memory material.

In one embodiment the shape memory material comprises a nitinol hypotube.

In another embodiment the anchoring element is encapsulated with an elastic polymer membrane that expands circumferentially when the anchoring system is compressed at deployment configuration.

In one embodiment, the coupling between at least one of the anchors and the linear actuator comprises a magnet or magnetisable element. This facilitates coupling of the anchor and linear actuator at a target location in-vivo. In this embodiment, the anchor external to the organ wall does not physically touch the linear actuator that is intra-luminal. One advantage of this embodiment is that it can obviate the need to penetrate the wall of the hollow organ.

In another embodiment, the system is designed for intraventricular placement, wherein the first anchor is adapted to engage a septum of a heart ventricle and a second anchor adapted to engage a free wall of the heart ventricle, and the electric linear actuator device is configured to extend and contract pulling the septum and free wall of the heart ventricle towards and away from each other repeatedly.

According to yet a further embodiment, the system designed for intraventricular placement includes a linear actuator configured to expand and contract to move the free wall of the heart towards the septum repeatedly.

In one embodiment, the electromagnetic linear actuator is configured to effect rotational actuation of the armature (i.e. the armature rotates during linear actuation). When the system comprises two electromagnetic linear actuators, one or both actuators may be configured to effect rotational actuation of the armature. The electromagnet may be configured for rotation of the armature of the electromagnet by up to 30 degrees, for example 5-30, 5-20, or 5-15 degrees. This forces one of the armatures to affect a rotational pull on the wall of the organ to simulate twisting movement. This may be achieved by employing a rotational electromagnet having a threaded armature disposed within a threaded housing. In one embodiment, the other of the electromagnetic linear actuators comprises a linear electromagnet. This is especially useful for systems for use in the heart, especially the left ventricle of the heart.

In another embodiment, the twisting action is achieved using magnetic couplings.

In one embodiment, the linear actuator is configured to exert a combined compression force on the ventricle of 1-15 Newtons, 5-15 Newtons, 1-5 Newtons, 5-10 newtons, 2.5-5 Newtons, or about 7-9 Newtons.

In one embodiment, the linear actuator is configured to contract and expand by about 5-11 mm, about 12-16 mm, about 13-15 mm, or about 14 mm, when actuated.

In one embodiment, the system has a diameter of 5-10 mm, typically 7-9 mm. In one embodiment, the system is configured for delivery in a 22 Fr delivery catheter. In one embodiment, the system has a length of 40-60 mm, 45-55 mm, and preferably about 50 mm (expanded form).

In one embodiment, the intraventricular assist device is configured for mounting in the left ventricle of the heart between a left ventricular mid-septum and left ventricle apex.

In another embodiment, the intraventricular assist device is configured for mounting in the right ventricle of the heart between a right ventricular mid-septum and right ventricle apex.

In one embodiment, the device comprises an inductive charging apparatus operatively coupled to the linear electromagnetic actuators.

In one embodiment, the device comprises a sensor configured to sense electrocardiogram information from the heart and a controller operatively connected to the sensor and the linear actuator, in which the controller is configured to actuate the linear electromagnetic actuators simultaneously according to information received from the sensor.

In one embodiment, one or both anchors may be configured for detachable coupling with the linear actuator. This allows one or both anchors to be delivered to the target location separately from the linear actuator. In one embodiment, the device is delivered in two parts, a first part comprising a first anchor, and a second part comprising the linear actuator coupled to the second anchor. For example, a distal anchor could be delivered to an external wall of the ventricle, and anchored in place, and then the second part (linear actuator coupled to proximal anchor) could be delivered separately to the target location, and the device assembled in-situ. In one embodiment, the distal anchor could be delivered apically (i.e. through the ribs by minimally invasive surgery), and the second part could be delivered percutaneously and trans-septally.

In another aspect, the invention provides a method of using an interventricular assist device of the invention, comprising the steps of:
delivering the device to a ventricle of the heart;
anchoring a first anchor and optionally a second anchor to a septum of the ventricle; and
actuating the linear actuator to expand and contract to move the septum and wall of the heart towards and away from each other repeatedly and thereby mechanically assist the ventricle in displacement of blood from the ventricle.

In one embodiment, the device is delivered to the left ventricle transluminally and trans-septally.

In one embodiment, the first anchor is anchored to the left ventricle mid-septum, and the second anchor is anchored to the apex of the left ventricle.

In one embodiment, the device is delivered to the right ventricle transluminally and trans-septally.

In one embodiment, the first anchor is anchored to the right ventricle mid-septum, and the second anchor is anchored to the apex of the right ventricle.

In one embodiment, the second anchor is anchored first, the device is then retracted, and the first anchor is then anchored.

In one embodiment, the method is employed to treat heart failure, especially left heart failure. In one embodiment, the method is employed to improve left ventricular function. Left ventricular function may be improved by one or more of: strengthening cardiac contraction; reducing left ventricular end diastolic pressure; reducing left ventricular wall tension; decreasing pulmonary congestion; and reducing pulmonary arterial pressure.

In an embodiment, the method is employed to treat STEMI patients with Left ventricular anterior wall akinesia/hypokinesia.

In a further embodiment, the method is employed to treat right heart failure. In one embodiment, the method is employed to improve right ventricular function.

In yet a further embodiment, the method is employed to treat heart failure that involves both sides of the heart. In one embodiment, the method is employed to improve both right and left ventricular function.

In one embodiment, the method employs a device in which one or both anchors may be configured for detachable coupling with the linear actuator and, prior to delivery, the first anchor is separate from the linear actuator, and the second anchor is attached to the linear actuator. In this embodiment, the method typically comprises the steps of: delivering the first anchor to the ventricle trans-septally and anchoring the first anchor in the ventricular septum with a distal end of the anchor projecting into the ventricle; delivering the linear actuator and second anchor to the ventricle apically; anchoring the second anchor to the wall of the ventricle; attaching the distal end of the linear actuator to the distal end of the anchor; and actuating the linear actuator to expand and contract to move the septum and wall of the heart towards and away from each other repeatedly and thereby mechanically assist the ventricle in displacement of blood from the ventricle.

In a further embodiment, the system is designed for intragastric placement, wherein the first anchor is adapted to engage one wall of the stomach and a second anchor adapted to engage another wall of the stomach, and the electric linear actuator device is configured to expand and contract to move the walls of the stomach towards and away from each other.

According to yet a further embodiment, the system designed for intragastric placement includes an electromagnetic linear actuator configured to expand and contract to move one wall of the stomach towards the other wall.

In one embodiment the system designed for intragastric placement includes an electromagnetic linear actuator configured to expand and contract to move the upper walls of the stomach towards each other.

In one embodiment the system designed for intragastric placement includes an electromagnetic linear actuator configured to expand and contract to move the lower walls of the stomach towards each other.

In one embodiment, at least two electromagnets are implanted in the stomach and are configured to effect sequential actuation of the armature such that the stomach contracts in sequence to generate a peristaltic wave that propels chyme in an anterograde direction.

According to a further embodiment, the system designed linear electromagnetic actuator is configured for anchoring to a proximal and a distal segment of the stomach at two opposing walls horizontally with configured diagonal angles. Once the actuators are engaged it would simulate the Migrating Myoelectric Complex (MMC) contraction sequences whose function is to sweep any residuals out of the GI tract. The linear actuators at the distal segment of stomach propagate at a slightly faster velocity of contraction in relation to the proximal actuators where contractions propagate more slowly.

In one embodiment, the system is designed for intragastric placement, wherein the first anchor is adapted to engage to the proximal segment of the stomach across from the opposing wall where the second anchor is engaged. The electric linear actuator device is configured to extend and contract, pulling the gastric walls of the stomach towards and away from each other repeatedly at specific travel distance, speed and force parameters.

In one embodiment, the linear actuator is configured to exert a combined compression force on the ventricle of 1-15 Newtons, typically 2-2.5 Newtons.

In one embodiment, the linear actuator at the proximal segment of the stomach is configured to contract and expand the linear actuator such that contractions propagate more slowly (3 contraction/minute and at a distance of <1 cm/sec).

In one embodiment, the linear actuator at the distal segment of the stomach is configured to contract and expand the linear actuator to provide a slightly faster velocity of contraction (traveling 3-4 cm/sec).

In one embodiment, the system has a diameter of 11-14 mm. In one embodiment, the system is configured for delivery in a 34 Fr delivery catheter. In one embodiment, the system has a length of 50-55 mm, and preferably about 80-100 mm (expanded form).

In one embodiment a non-invasively monitoring device at the exterior of the stomach would detect electrical signals that travel through stomach muscles which control gastric contractions. These signals will than trigger the actuators contraction sequence.

According to an additional aspect of the present invention, there is provided an intragastric contraction assist device comprising: a first anchor adapted to engage a first wall of the gastrointestinal tract; a second anchor adapted to engage a second wall of the gastrointestinal tract; and a linear actuator coupled with the first anchor and second anchor, and configured to expand and contract to provide relative reciprocating movement between the first and second walls.

In one embodiment, the second wall is disposed opposite the first wall.

In one embodiment, the first and second wall are disposed within the same section of the gastrointestinal tract (i.e. both are walls of the stomach, or the intestine).

In one embodiment, the first wall is disposed within a first section of the gastrointestinal tract, and the second wall is disposed in a second section of the gastrointestinal tract (ideally an adjacent section).

According to a further aspect of the present invention, there is provided a urinary bladder contraction assist device comprising: a first anchor adapted to engage a first wall of the urinary bladder; a second anchor adapted to engage a second wall of the urinary bladder; and a linear actuator coupled with the first anchor and second anchor, and configured to expand and contract to provide relative reciprocating movement between the first and second walls.

In an additional embodiment, the system is designed for intra-bladder placement, wherein the first anchor is adapted to engage one wall of the urinary bladder and a second anchor is adapted to engage another wall of the urinary bladder, and the electric linear actuator device is configured to expand and contract to move the walls of the urinary bladder towards and away from each other.

In one embodiment, the linear actuator is configured to exert a combined compression force on the bladder of 1-15 Newtons, 5-15 Newtons, 1-5 Newtons, 5-10 newtons, 2.5-5 Newtons, or about 7-9 Newtons.

In one embodiment, the linear actuator is configured to contract and expand by about 5-120 mm, about 10-110 mm, about 25-100 mm, or about 100 mm, when actuated.

In one embodiment, the linear actuator has an expanded intraluminal (intra-bladder) length of 50-250 mm, 60-200 mm, and preferably about 120 mm (expanded form).

In one embodiment, the system has a contracted intraluminal (intra-bladder) length of 20-80 mm, 30-70 mm, and preferably about 50 mm.

In one embodiment, during the non-contracting phase of the linear actuator, the intraluminal (intra-bladder) linear actuator exerts a pulling force that is close to 0 Newtons (thereby minimizing any hindrance of the ability of the bladder to distend during the urine-filling phase).

In one embodiment, a sensor measures the flow rate of the expulsion of urine and adjusts the output parameters (such as force, speed or travel) of the linear actuator such that the flow rate of expelled urine is maintained between 20 to 30 ml/s.

In another embodiment, a sensor measures the level of stretch of bladder such that a signal is transmitted to the control system indicating the level of bladder fullness.

In yet another embodiment, a pressure transducer measures the intra-bladder pressure to such that a signal is transmitted to the control system indicating the level of bladder fullness.

In one embodiment, a pressure sensor measures the intravesical pressure of the bladder and adjusts the output parameters (such as force, speed or travel) of the linear actuator such that intravesical pressures between 50-60 cmH2O are achieved during micturition (urination) through assisted contraction of the bladder walls.

In one embodiment, the pressure sensor is located outside the bladder and measures the intravesical pressure of the bladder and adjusts the output parameters (such as force, speed or travel) of the linear actuator such that intravesical pressures between 50-60 cmH2O are achieved during micturition (urination) through assisted contraction of the bladder walls.

In one embodiment, the pressure sensor is located within the bladder and measures the intravesical pressure of the bladder and adjusts the output parameters (such as force, speed or travel) of the linear actuator such that intravesical pressures between 50-60 cmH2O are achieved during micturition (urination) through assisted contraction of the bladder walls.

In one embodiment, a sensor measures the intravesical volume of urine within the bladder and adjusts the output parameters (such as force, speed or travel) of the linear actuator such that intravesical volume of urine within the bladder can be reduced once these volumes are above, say, 200 ml or 300 ml or 400 ml or 500 ml.

In another embodiment, the duration of the contraction force applied by the linear actuation system is between 5 seconds and 120 seconds.

In another embodiment, the invention provides a method of using the device of the invention, comprising the steps of: delivering the device in to the bladder; anchoring a first anchor to a wall of the bladder; anchoring a second anchor to an opposing wall of the bladder; and actuating the linear actuator to expand and contract to move the bladder walls towards and away from each other and thereby mechanically assist the bladder in displacement of urine from the bladder.

In one embodiment, the device is delivered to the bladder through a transurethral catheter.

In another embodiment, the device is delivered to the bladder surgically, through an incision, or several incisions in the bladder wall.

In one embodiment, one or more anchors may be configured for detachable coupling with linear actuators. This allows the anchors to be delivered to the target location separately from the linear actuator.

In one embodiment, the device is delivered in two steps, a first step comprising anchors, and a second comprising the linear actuator. For example, anchors are delivered to external walls of the bladder via a minimally invasive surgical procedure. Subsequently the linear actuation system is delivered to the target location via the urethra. The device is assembled in-situ by connecting the anchors to the linear actuation system.

In one embodiment, the coupling between at least one of the anchors and the linear actuator comprises a magnet or magnetisable element. This facilitates coupling of the anchor and linear actuator at a target location in-vivo. This electromagnetic coupling can be solely used for delivery purposes or can also be the final implanted configuration.

In another embodiment, the anchor and the linear actuator would not actually be physically in contact but are held in close proximity through electromagnetic fields that exist either side of the bladder wall.

The programmable intra-luminal contraction augmentation system may be an electrical medical system. The system may include a real-time operating system. The system may include an embedded platform for automation. The system may include firmware software components. The system may also include an application specific integrated circuit (ASIC), a Programmable Logic Device (PLD) which may include digital circuits, a digital signal processor, a microcontroller or a microprocessor, a memory component and a controller circuit.

The system may include analog interfaces (digital-to-analog, analog-to-digital). The system may include voltage regulators and power management circuits. The system may additionally include timing sources. Examples of timing sources that may be used include phase lock loop control systems and oscillators.

Other aspects and preferred embodiments of the invention are defined and described in the other claims set out below.

Additional beneficial embodiments and combinations of aspects of the invention are described in detail below.

### BRIEF DESCRIPTION OF THE FIGURES

FIG. 1 is a perspective view of a first embodiment of a linear actuator forming part of the contraction device of the system of the invention;

FIG. 2 is a perspective view of one of the linear solenoids forming part of the contraction device of FIG. 1 showing the armature attached to the coil housing;

FIG. 3 is an exploded view of the linear solenoid of FIG. 2 showing the armature detached from the coil housing;

FIG. 4 is a perspective view of the contraction device showing the distal and proximal anchors attached to the device;

FIG. 5 is a perspective view of a second embodiment of a linear actuator forming part of the contraction device of the system of the invention;

FIG. 6 is a perspective view of a rotational solenoid forming part of the contraction device showing the helical threads on the armature;

FIG. 7 is an illustration of a human heart showing one exemplary configuration of the contraction device implanted in the left ventricle between the left ventricle mid-septum and apex of the left ventricle;

FIG. 8 shows another exemplary configuration of the contraction device being deployed for use with the heart corresponding to an across left ventricle location;

FIG. 9 shows another exemplary configuration of the contraction device being deployed for use with the heart corresponding to a diagonal right ventricle location;

FIG. 10 shows a block diagram of one embodiment of the main components of the system of the invention when deployed for use in the heart;

FIG. 11 illustrates the contraction device deployed in the heart with the pressure sensor and power unit implanted outside the heart;

FIG. 12 illustrates the contraction device deployed in the heart with the ECG array and power unit implanted outside the heart;

FIG. 13 is an illustration of a stomach showing the contraction device implanted in the pyloric antrum;

FIG. 14 is an illustration of a stomach showing the contraction device implanted in the mid-antrum;

FIG. 15 is an illustration of a stomach showing the contraction device implanted in the upper fundus;

FIG. 16 shows a block diagram of one embodiment of the main components of the system of the invention when deployed for use in the stomach;

FIG. 17 illustrates the contraction device deployed in the stomach with the pressure sensor and power unit implanted outside the stomach;

FIG. 18 is an illustration of a bladder showing the contraction device implanted between the base and the mid superior surface of the bladder;

FIG. 19 is an illustration of a bladder showing the contraction device implanted between the two infero-lateral walls of the bladder;

FIG. 20 is an illustration of a bladder showing the contraction device implanted between the lower-left and right infero-lateral wall and the superior surface of the bladder;

FIG. 21 shows a block diagram of one embodiment of the main components of the system of the invention when deployed for use in the bladder;

FIG. 22 illustrates the contraction device deployed in the bladder in communication with the user activated external control unit; and

FIG. 23 shows a block diagram of another embodiment of the main components of the system of the invention when deployed for use in the bladder.

### DETAILED DESCRIPTION OF THE INVENTION

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

Additionally, the terms “treatment” or “treating” refers to an intervention (e.g. the use of the device of the invention to assist systole of the left ventricle) which prevents or delays the onset or progression of a disease or reduces (or eradicates) its incidence within a treated population. In this case, the term treatment is used synonymously with the term “prophylaxis”.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

“Electromagnetic linear actuators” refers to electric linear actuators that convert electrical energy into mechanical energy through the interaction of magnetic fields and current-carrying conductors to generate force. They operate through the electromagnetic principle for energy conversion to transform electric current into linear motion.

“Linear solenoid” or “linear solenoid actuator” refers to an electromagnetic device that converts electrical energy into a mechanical pushing or pulling force or motion. The device generally comprises an electrical coil wound around a cylindrical tube and a ferromagnetic actuator (armature) that is free to slide in and out of the coil body. The solenoid is generally configured such that upon actuation, the armature is retracted into the housing due to the magnetic flux generated by the coil upon actuation. The force and speed of the armature retraction is determined by the strength of the magnetic flux generated within the coil. Upon removal of the magnetic flux, the armature returns to an extended position. In some linear solenoid designs, this is achieved by means of a return spring coupled to the armature.

There are various configurations of linear solenoids and several approaches to control their function. Examples include the latching solenoid, the Peak and Hold Solenoid, the Proportional Solenoid and Pulse-Width Modulation (PWM).

“Latching solenoid” means a solenoid that uses a permanent magnet and is used for applications requiring sustaining an open or closed position for prolonged periods of time.

“Peak and Hold solenoids” means a solenoid that uses higher current for peak load function and lower current for holding function, thus allowing fast actuation, low power consumption and low heat production.

“Proportional solenoid” means a solenoid that reduces friction via utilization of bearings to generate low hysteresis and produce flat force versus stroke curves. These features allow accurate positioning and force control. PWM solenoids apply voltage at specified frequencies to vary the force produced by the solenoid. Frequencies of proportional current control generally range between 200-1000 Hz. Frequencies of digital current control generally range between 25-200 Hz.

“Rotational solenoid” or “rotary solenoid” refers to a form of linear solenoid actuator in which the armature is forced to rotate during extension and retraction.

“Back to back linear solenoids” refers to two linear solenoids connected back-to-back and having an armature extending from each free end thereof. As the solenoids are connected back-to-back, the retraction of the armatures of the respective solenoids causes a reduction of the effective length of the device. It will be appreciated that the back-to-back solenoids may be a provided by a single unitary device, or by two separate solenoids that are coupled together, for example by adhesive, welding or another fixing means. One of the solenoids may be a linear latching solenoid and the other may be a rotational solenoid.

“Moving coil linear actuator” means an actuator that comprises a permanent magnet frame and a mobile coil driven by a current. The coil is placed into a magnetic field perpendicular to the coil winding. When electric current is applied to the wire coil it creates a magnetic field that interacts with the permanent magnetic field to produce linear movement of the coil perpendicular to the direction of the current. The force of coil movement generated is a Lorentz force and is directly proportional at any position in the stroke to the amount of current applied to the coil. Moving coil actuators can move bidirectionally and produce a relatively constant force over the entire stroke.

“Moving magnet actuator” means an actuator that comprises a mobile permanent magnet placed between two opposite electromagnetic poles that are activated by passing a current through a fixed coil assembly. The moving magnet assembly moves when current is applied to the fixed coil. Moving magnet actuators are direct-drive electromagnetic linear actuators generating frictionless and non-recoil motion over a range of several millimetres. Force output of moving coil actuators is high. In an embodiment of the moving magnet assembly a permanent-magnetic-field assembly piston moves inside a cylindrical coil tube.

In a further embodiment the moving magnet assembly includes a permanent magnet attached to a shaft and end caps that contain bearings.

“Organ Contraction Parameters” mean changes in organ size, organ dimensions, contraction timing, contraction duration, contraction sequencing, contraction frequencies, contraction strength, organ wall stress, organ wall thickness, organ wall tension, organ compliance, nerve activity, electrical activity, organ volume, organ pressure and volume of liquid in organ.

“Device output parameters” mean linear actuator stroke length, stroke direction, actuation force, torque, tension, compression, actuation pattern, linear velocity, actuation force curve, duty cycle, frequency, force vs. stroke performance, and combinations thereof.

“Anchor” means an element configured for engaging tissue, for example a wall of hollow lumen such as a wall or septum of a heart chamber, gastrointestinal organ, or bladder.

“Magnetic Coupling” means a coupling that transfers torque from one shaft to another using a magnetic field or electromagnetic field rather than a physical mechanical connection.

“Inductive charging apparatus” means an apparatus for charging the device wirelessly by using any one of electromagnetic field, wireless radio waves or magnetic resonance charging to transfer energy to charge the electronic device.

The present invention provides an implantable intraluminal contraction augmentation (ICA) system. The system comprises at least one implantable organ contraction device for producing a contraction force, an anchoring assembly for implanting the at least one contraction device in a hollow contractile organ of the body, and a controller for modifying the output parameters of the contraction device. In one embodiment, the system also comprises at least one sensor in communication with the controller for detecting contraction parameters associated with the contractile organ. The controller is configured to modify the output parameters of the contraction device based on the sensor data.

The system of the invention is designed to deliver clinically effective functional support to the implanted hollow organ. The system increases the output of the hollow contractile organ in which it is implanted. Organs that may be implanted with the contraction device include the heart, oesophagus, stomach, intestines and urinary bladder, where the system is configured to provide a clinically significant increase in ejection of blood, urine, and chyme respectively. The level of contraction assistance is adapted to each of the organs. For example, the device may be placed in the left ventricle (LV) of the heart, where it will assist LV systolic contraction, thereby increasing cardiac output in systolic heart failure patients.

The controller is programmable such that the system can act as an auto-adaptive system that responds to physiologic cues. The system can continuously adapt to specific user requirements. The contraction device is configured by means of the controller to activate in a pattern synergistic to the natural contraction cycle of the contractile organ.

For example, when implanted in the heart, the contraction device may be configured to respond to the rate of contraction of the heart and activate in sync with the contraction of the myocardium as detected by the one or more sensors. Thus, the contraction device is configured to activate during systole and halt operation during diastole. Furthermore, when demands on the heart output are detected to have increased, the controller can modify the output parameters of the contraction device so as to generate an increased actuation force output and modify the activation frequency of the contraction device to meet the increase in heart rate. Conversely, when heart output demands are detected to have reduced, the controller can modify the output parameters of the contraction device to generate a reduced actuation force output and change its activation frequency to be synchronised with the reduced heart rate frequency.

In the stomach, the contraction device is configured to respond to the natural contraction activity of the stomach and may be activated when one or more sensors detect contraction of the pyloric antrum or pyloric canal regions of the stomach. It may also be activated in the stomach when one or more sensors detect that there is chyme in the stomach beyond a specific volume. The contraction device may deactivate automatically once the volume of chyme is reduced.

In most embodiments of the invention, such as when the system is for use with the heart or stomach, the controller modifies the output parameters of the contraction device based on organ contraction parameters detected by the at least one sensor. However, when the system is for use with the bladder, the controller receives an external input signal from a user to activate the contraction device, and may not include a sensor. This is because activation of the contraction device should occur only when the user is ready to urinate. In this, case, the user receives sensory input from the nervous system and naturally senses when the bladder is full. The user can then activate the contraction device to generate a contraction of the bladder resulting in urination by activating an external control unit in communication with the controller.

In an alternative embodiment for use with the bladder, the system is configured to accommodate a patient who is unable to sense a full bladder. In this embodiment, the system also includes a sensor. The sensor detects when the bladder is full, and in response sends an alert to the user to activate the contraction device manually via activation of the external control unit in communication with the controller. This alert may take any suitable form known to a person skilled in the art. The controller can then deactivate the linear actuation functions of the contraction device once intra-bladder pressure drops below a predetermined threshold value.

In one embodiment of the invention, the contraction device comprises an electromagnetic linear actuator. Electromagnetic linear actuators are known to have many advantages over other types of actuators including fast response and simple control laws. Moreover, whereas the accuracy of hydraulic and pneumatic actuators is limited by the compressive properties of liquid and gas, electric actuators possess high positioning accuracy and are employed in high precision, high speed applications. Linear electromagnetic actuators are ideally suited for use with intraluminal devices due to their size, the compression forces that can be obtained, easier implantation compared with large extra ventricular braces, low power usage, and their ability to generate actuation with high acceleration and fast actuating speed due to their non-contact and frictionless characteristics. Additionally, they are suitable for being powered by a battery (which can be housed within the system) or by means of inductive charging, thereby obviating the need to couple the contraction device to an external power source. Electromagnetic linear actuators are also suitable for providing both linear and rotational actuation, which makes them particularly useful for use in contractile hollow organs. Furthermore, as electromagnetic actuators do not rely upon hydraulic liquids or compressed air, there is reduced risk of a foreign liquid or pressurised air leaking or bursting within the contractile organ. A further advantage is that electromagnetic actuators obviate the requirement for a reservoir of air or hydraulic fluid, and associated pump.

Examples of linear electromagnetic actuators that are suitable for use as the contraction device include moving magnet, moving coil and moving iron, and solenoid actuators. The electromagnetic linear actuator selected for use with a particular organ is based on specific testing to determine which electromagnetic linear actuator type and configuration is most suited to provide a clinically significant output increase over time in a safe and repeatable manner for the organ. For example, in some cases it would be advantageous to use an electromagnetic actuation system that generates a flat force curve.

The contraction device may comprise at least one electromagnetic actuator, for example two or three electromagnetic actuators. When two electromagnetic actuators are being used, they may be connected together in a number of different arrangements. For example, they may be connected as back-to-back electromagnetic actuators, positioned one on top of the other, positioned parallel to each other, or positioned perpendicular to each other. For example, they may be positioned at an angle above 0 degrees and less than or equal to 90 degrees to each other. Two or more electronic linear actuators may also be connected via a tethering means.

When the system is to be used with particular organs, it is advantageous to use an electromagnetic actuator that can generate torque in addition to axial force. For example, the left ventricle of the heart, during systole, compresses and twists. The linear solenoid actuator may be configured to affect a rotational movement of its armature during actuation (i.e. the armature rotates during retraction and extension), with a controllable degree of rotation. This can be achieved by having cooperating threads on the armature and cylindrical tube which force the armature to rotate during retraction and extension. The solenoid may be configured for rotation of the armature by up to 30 degrees during retraction or extension, for example 5-30, 5-20, or 5-15 degrees.

When the contraction device comprises two electromagnetic linear actuators, one or both of the actuators may be configured to effect rotational actuation of the armature of the electromagnet. In another embodiment, the twisting action is achieved using magnetic couplings.

In one embodiment of the invention, at least part, and preferably all, of the actuator is disposed within a fluid impermeable biocompatible sleeve to protect the actuator from fluid in the organ (such as blood in the heart). The sleeve may be fabricated from any suitable material, such as for example a silicone capsule. The one or more distal ends of one or more actuator armatures may be exposed proud of the end of the sleeve. The armature can be provided with a circumferential groove such that the sleeve termination sits inside the groove and is fixed within the groove.

In one embodiment, the sleeve is a non-compliant foldable structure which is configured to unfold to accommodate expansion of the actuator. In another embodiment, the sleeve is compliant, and configured to stretch during expansion of the actuator. Accordingly, the sleeve is in a stretched state when the armatures of the actuator are fully extended, providing additional potential contraction energy during the contraction phase of the actuator.

The sleeve may also include a bellow arrangement outside the armatures to allow movement of the sleeve. The natural physical disposition of the bellow is such that in its expanded form, when the armatures are fully extended, the bellow provides additional potential contraction energy during the contraction phase of the linear actuator.

The anchoring assembly comprises anchors that are configured to be deployed and embedded in the tissue of the wall of an organ in which the contraction device is to be implanted to ensure a secure attachment of the contraction device. Anchoring is usually at a minimum of two points in the organ. The contraction device is designed to pull on the anchors to cause contraction of the anchored organ walls.

The anchors are adapted to withstand repeated loads generated by both the contraction device, as well as loads generated by the contractile tissue in which they are embedded. In addition, the anchors are designed to minimise damage to the surrounding tissue, through designs that minimise the damage caused through penetration of the anchor through muscle tissue, and any subsequent damage to the tissue generated by repeated and continuous contraction cycles. For example, an anchor having a wing design with an elastic polymer membrane prevents damage of the muscle tissue, as it distributes pulling contraction force into a circumferential area of the wings.

The anchor may be formed from any suitable structure. Anchors are selected according to tissue type and the condition of the tissue. For example, for anchoring into the thick tissue of the ventricle septum, an anchor may require two wing elements, while for anchoring into the thinner tissue of the urinary bladder, an anchor may only require a single wing element.

In one embodiment, the anchor is encapsulated with an elastic polymer membrane that expands circumferentially when the anchoring assembly is compressed at deployment configuration.

In one embodiment, the coupling between at least one of the anchors and the contraction device comprises a magnet or magnetisable element. This facilitates coupling of the anchor and the contraction device at a target location in-vivo. In this embodiment, the anchor external to the organ wall does not physically touch the contraction device that is intra-luminal. One advantage of this embodiment is that it can obviate the need to penetrate the wall of the hollow organ.

In one embodiment, the anchors are configured for detachable coupling with the contraction device. This allows one or both anchors to be delivered to the target location separately from the contraction device. In one embodiment, the device is delivered in two parts, a first part comprising a first anchor, and a second part comprising the contraction device coupled to the second anchor. For example, a distal anchor could be delivered to an external wall of the organ, and anchored in place, and then the second part (contraction device coupled to proximal anchor) could be delivered separately to the target location, and the device assembled in-situ. In one embodiment, the distal anchor could be delivered apically (i.e. through the ribs by minimally invasive surgery), and the second part could be delivered percutaneously and trans-septally.

The system also comprises a power unit for supplying power to the contraction device. The power unit may comprise a battery embeddable in the body that can be charged wirelessly using electromagnetic inductive charging. Charging energy is sent through an inductive coupling to the battery. In one embodiment, the inductive coupling may be achieved via a charging pad. The charging pad power cable is plugged into a wall outlet and the pad is placed inside a specially designed elastic chest belt with a pocket to place and hold the pad over the chest adjacent to the left lower rib cage for the period of charging.

Power may be supplied to the contraction device by a number of different arrangements. In one embodiment, power is provided via the anchoring assembly. This arrangement obviates the requirement to provide an incision or a hole separate to the anchoring holes in the organ (or chamber) wall specifically for supplying power to the contraction device. In an alternative embodiment, power supply to the contraction device is provided via a separate incision or hole in the organ (or chamber) wall specifically for power supply.

It will be appreciated that the selection of the one or more types of sensor for use in the system for sensing the contraction parameters of the organ is dependent on the organ in which the contraction device is to be implanted. For example, when the contraction device is to be used in a heart, the sensor may comprise a sensor configured to sense the electrocardiogram information from the heart. Examples of suitable sensors include pressure sensors, ventricle pressure sensors, wireless pressure ventricular pressure sensors, mems pressure sensors, artery pressure sensors, cardiac output (CO) sensors, blood pressure sensors, heart rate sensors, motion sensors, accelerometers, ECG (Electrocardiogram) sensors, O₂ saturation sensors, microaccelerometers, sonomicrometers, and real-time contractility sensors.

The system may further include telemetric components for transmitting and receiving signals relating to the activity of the implanted organ and the activity of the system. Telemetric components may include wireless medical telemetric elements using radio frequency to relay data such as pulse, heart rate, electrical activity of the heart, electrical activity of the stomach, electrical activity of the urinary bladder, and muscle contractility to the controller.

The controller may be configured to control the operation of the contraction device either through automatic execution of program instructions in memory and/or upon receiving an external input from a user. The memory component of the system may include ROM and RAM memory. The controller may take the form of a microprocessor.

### Exemplification

The invention will now be described with reference to specific examples. These are merely exemplary and for illustrative purposes only: they are not intended to be limiting in any way to the scope of the monopoly claimed or to the invention described.

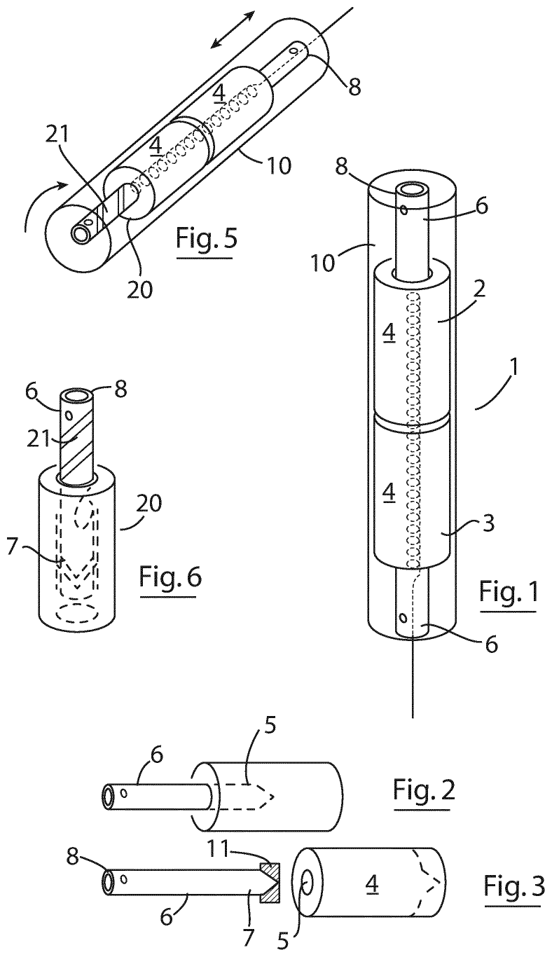

FIGS. 1 to 3 show the contraction device of the ICA system of the present invention when it is embodied as a linear actuator, shown without the distal and proximal anchors, and indicated generally by the reference numeral 1. The linear actuator comprises two back-to-back solenoids, namely a distal solenoid 2 and a proximal solenoid 3, which are attached together end to end. In this embodiment, each solenoid is the same and comprises a coil housing 4 having a length of about 12 mm and a width of about 5 mm, containing an electrical coil (not shown) wound around a cylindrical housing 5, an armature 6 having a length of 15 mm and a diameter of about 2 mm, and having a proximal end 7 disposed within the cylindrical housing 5 to a depth of about 5 mm, and a distal end 8 exposed proud of the coil housing by about 5 mm. Each armature is configured for retraction into the coil housing when the coil is electrically energised by means of power supplied via a cable extending through the armature to the coil, by a distance of about 7 mm. Thus, when both armatures are retracted into the respective coil housings, the effective length of the linear actuator reduces by about 14 mm. Each armature is configured to return to its starting extended position when current to the electrical coil is stopped. When the device is implanted in an organ, this can be achieved through the supply of power to the contraction device and also through the walls of the organ acting on the armature. However, in one embodiment, a return spring is used to achieve this function. In such an embodiment, the return spring is mounted over the armature 6 at its proximal end 7, encapsulated within the coil housing. The proximal end 7 of each armature comprises a shoulder formation 11 (FIG. 3) configured to prevent the armature being detached from the cylindrical housing 5. As shown in FIG. 1, the device comprises a silicone sleeve 10 which encapsulates the linear actuator 1 and has apertures at each end to allow the distal ends 8 of the armature to extend beyond the sleeve.

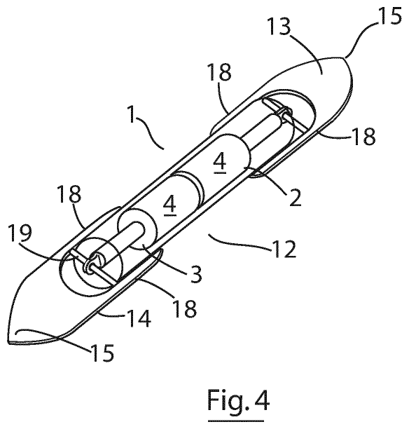

FIG. 4 shows one embodiment of the anchoring assembly of the system attached to the contraction device. In this embodiment, two anchors are attached to the device 1, a distal anchor 13 attached to the distal end 8 of the armature of solenoid 2, and a proximal anchor 14 attached to the distal end 8 of the armature of solenoid 3. Each anchor 13, 14 comprises an anchoring plate having a piercing head 15 and two arms 18, and an axle 19 extending between the arms 18. However, it should be understood that the spear-shaped anchoring assembly of FIG. 4 is only one exemplary embodiment of an anchoring assembly, and any other suitable shape and form of anchoring assembly could equally well be used.

In the embodiment described above, the contraction device comprises a linear actuator which comprises identical linear solenoid actuators, which when actuated cause linear retraction of the armatures. FIGS. 5 and 6 illustrate an alternative linear actuator, in which parts identified with reference to the previous embodiments are assigned the same reference numerals, and in which one of the solenoids is a linear solenoid (proximal solenoid 3), and one of the solenoids is a rotational solenoid (distal solenoid 20). The rotational solenoid when actuated retracts and extends the same amount as the linear solenoid, and at the same speed and with the same force, but also rotates about 10-15 degrees during retraction and extension. Referring to FIG. 6, the armature of the distal solenoid 20 includes a helical thread 21 which during retraction and extension cooperates with a helical thread (not shown) formed on the inside of the cylindrical tube, which forces the armature to rotate during the retraction and extension. This forces the distal armature to affect a rotational pull (for example, 15-20 degrees) to simulate twisting movement when contracting. The delivery and use of this embodiment is substantially the same as that described with reference to the previous embodiment.

As previously explained, the implantable intraluminal contraction augmentation (ICA) system of the present invention can be used with a number of different contractile organs to augment the contraction of the organ.

For example, the contraction device of the system may be implanted in the left ventricle of the heart so that an anchor holds one electromagnetic pull linear actuator armature at the left ventricle free wall and another anchor holds another electromagnetic pull linear actuator armature at the apex of the left ventricle so that the lines of contraction of the two pull electromagnetic linear actuators are perpendicular to each other. In another example, the contraction device of the system may be implanted in the left ventricle of the heart so that an anchor holds one electromagnetic pull linear actuator armature at the left ventricle free wall and another anchor holds another electromagnetic pull linear actuator armature at the septum of the left ventricle so that the lines of contraction of the two pull electromagnetic linear actuators are collinear to each other.

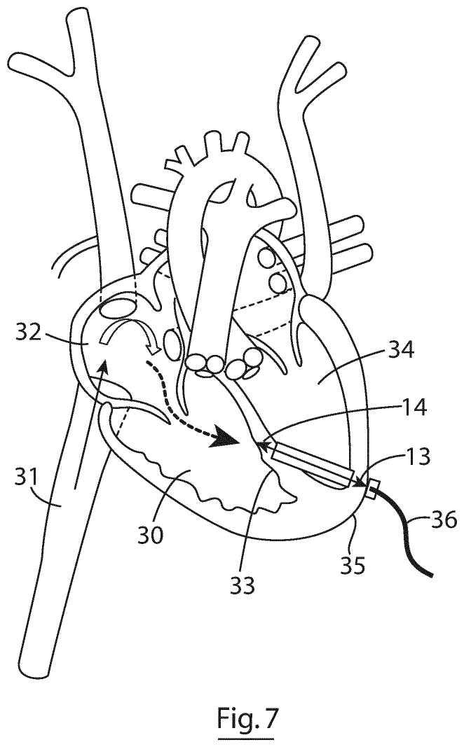

FIG. 7 shows the contraction device 1 being deployed for use with the heart in one exemplary configuration, corresponding to a diagonal left ventricle location. In accordance with one exemplary method for deployment in this location, the contraction device 1 is firstly percutaneously delivered to the right ventricle of the heart 30 via the vena cava 31 and right atrium 32. This may be achieved for example by means of a 22 Fr delivery catheter. The device is then advanced to the right ventricle, and the septal wall can be punctured using standard available equipment and known techniques. The device will be advanced through the septal wall 33 aperture into the left ventricle 34. The septal anchors will hold the device on the septal side. In the left ventricle 34, the distal anchor 13 is embedded into the wall at the apex 35 of the left ventricle. The device is then retracted by the surgeon towards the left ventricle mid-septum 33, where the proximal anchor 14 is embedded into the mid-septum of the left ventricle. The device is now implanted as shown in FIG. 7, spanning the septum and apex of the left ventricle. In another exemplary method for deployment to the diagonal left ventricle, the contraction device 1 is delivered trans apically by means of a minimal surgical insertion through the rib cage into the left ventricle via the septum of the heart.

As shown in FIG. 7, a lead 36 may be provided through the distal armature and anchor for electrically connecting the device to a sensor (not shown) and the power unit (not shown).

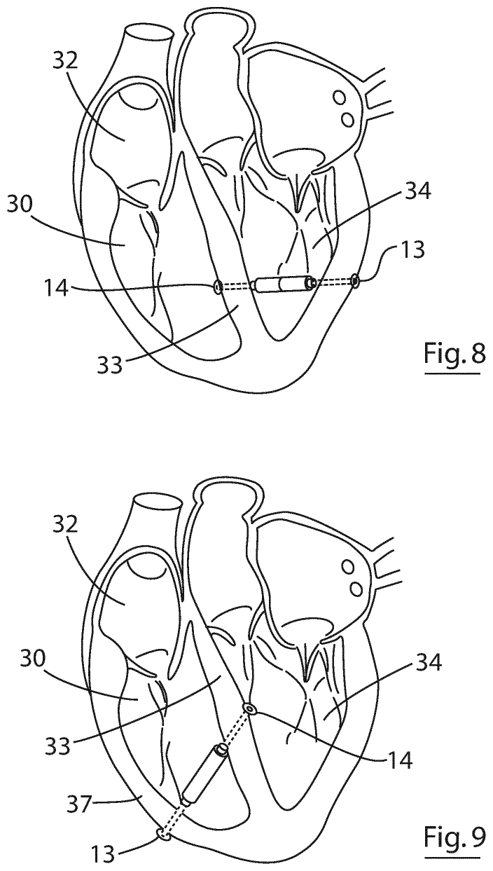

In the embodiment described in FIG. 7, the contraction device is implanted in the left ventricle between the left ventricular mid-septum and left ventricle apex. FIG. 8 shows the contraction device 1 being deployed for use with the heart in another exemplary configuration, corresponding to an across left ventricle location. Accordingly, the contraction device of FIGS. 7 and 8 is employed to improve left ventricular function by one or more of strengthening cardiac contraction, reducing left ventricular end diastolic pressure, reducing left ventricular wall tension, decreasing pulmonary congestion, and reducing pulmonary arterial pressure.

FIG. 9 shows the contraction device 1 being deployed for use with the heart in yet another exemplary configuration, corresponding to a diagonal right ventricle location, in which the contraction device is mounted in the right ventricle of the heart between a right ventricular mid-septum and right ventricle apex. In this embodiment, the contraction device of the invention treats right heart failure by improving right ventricular function.

It will further be appreciated that in another embodiment of the invention, the ICA system of the invention could be used to treat heart failure that involves both sides of the heart by appropriate implantation of the contraction device in the heart.

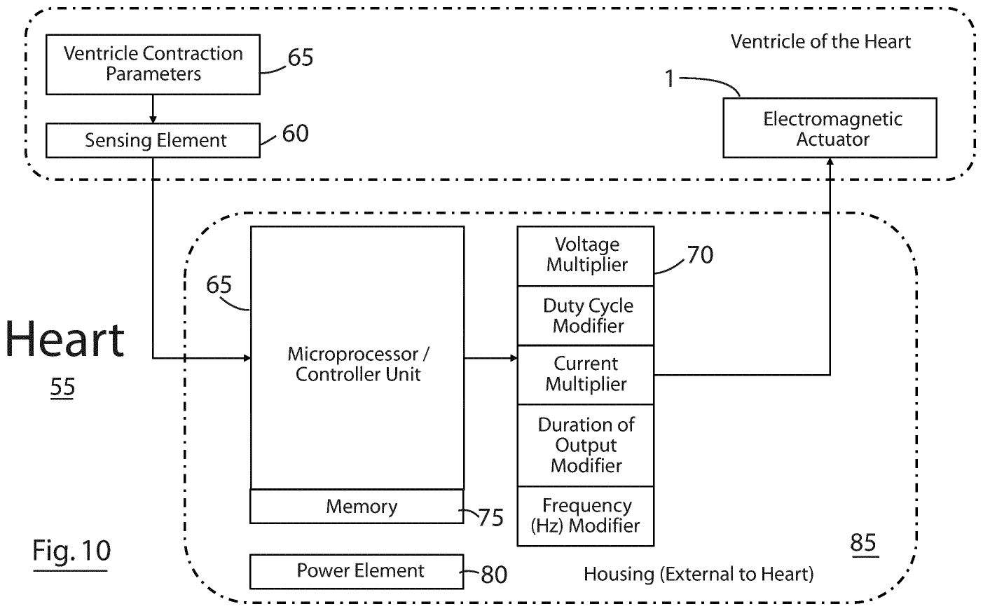

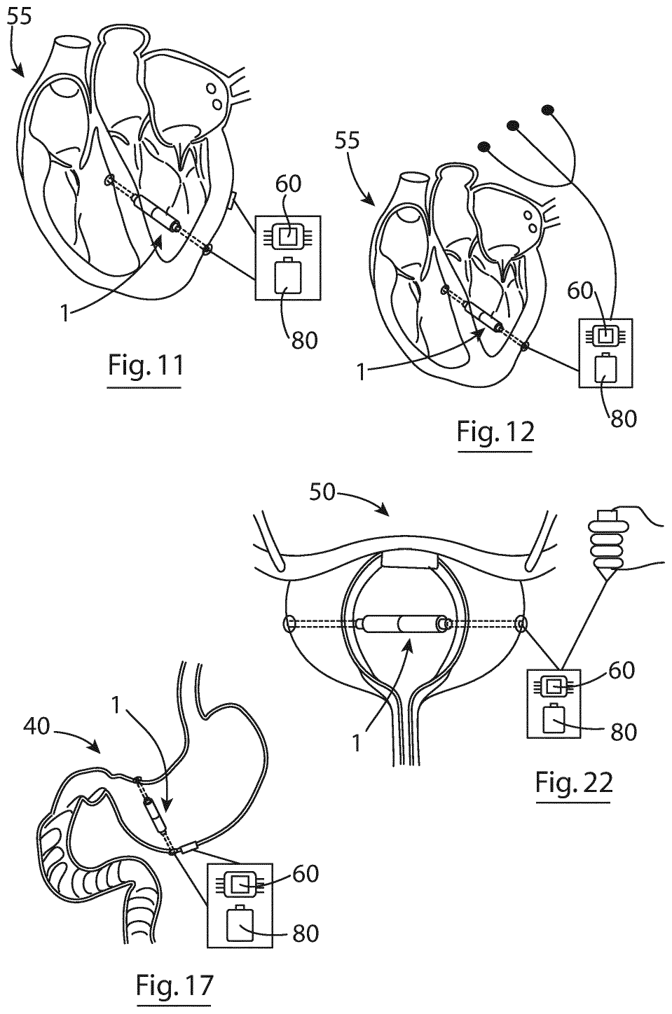

FIG. 10 shows a block diagram of one embodiment of the main components of the system of the invention when deployed for use in the heart. In the embodiment shown in this figure, both the contraction device 1 in the form of an electromagnetic actuator 1 and the sensor 60 are located in the ventricle of the heart 55. The sensor 60 is configured to detect ventricle contraction parameters 65. In one embodiment, the sensor 60 comprises a pressure sensor located on the anchor assembly and positioned on the ventricle wall. A sensor may additionally or alternatively be provided external to the heart. FIG. 11 shows such an example in respect of a pressure sensor and the power unit, while FIG. 12 shows such an example in respect of an ECG array and the power unit. The ECG array has one or more leads that are placed on the skin of the user, as shown in FIG. 12. The ECG leads may be positioned subcutaneously or implanted, such as for example in the left parasternal region of the body.

The controller 65 is located external to the heart. In the embodiment shown in FIG. 10, a housing 85 houses the controller 65, as well as electronic circuitry 70 associated with the contraction device for setting the output parameters of the electromagnetic actuator 1 in accordance with control signals received from the controller. In the embodiment shown, the electronic circuitry comprises a voltage multiplier, a duty cycle modifier, a current multiplier, a duration of output modifier and a frequency modifier. Memory 75 and power unit 80 associated with the electronic circuitry are also housed in the housing 85. The housing 85 may be positioned in any suitable location on the body. Examples of suitable locations include the thoracic cavity, the left hypochondriac region, and under the skin (in the left chest area). The housing 85 may also be located in the left shoulder area, by means of an incision made below the collar bone.

The sensor data output from the sensor 60 is communicated to the controller 65 by means of either wired or wireless communications. The controller 65 is configured to continuously assess the time of endogenous ventricular depolarization and contraction based on the received sensor data and optimally synchronise the activity of the implanted linear actuator 1 to the endogenous cardiac cycle by modifying the output parameters of the actuator 1.

In use, the sensor 60 detects the electrical activity of the heart (e.g. ventricle contraction or depolarization), and sends the sensor data to the controller 65. The received sensor data is analysed by the controller 65 according to the instructions stored in the memory 75. The controller 65 then delivers control signals to the power unit 80 and to the electronic circuitry 70 so as to modify the output parameters of the contraction device 1 as appropriate based on the sensor data so as to activate the contraction device 1 in a pattern synergistic to the natural contraction cycle of the heart.

It should be understood that some of the output parameters of the contraction device are determined by the predefined structural and technical specifications of the linear actuator, such as for example the maximal force output, the capability to general torque, and compression versus tension. However, other output parameters of the contraction device are controllable. These include for example actuation frequency, actuation force, actuation acceleration rate and travel distance of actuation armature. Such output parameters are controllable in real time by the controller 65 being configured to modify the parameters associated with the contraction device such as duty cycle, current, timing and frequency of current flow by sending appropriate control signals to the electronic circuitry 70 associated with the electromagnetic actuator 1 and the power unit 80.

When the controller 65 is configured to energise the solenoids of the contraction device 1 when the sensor 60 detects the systole stage of a heart beat, it causes the armatures of the two solenoids to be retracted into the coil housing, which, when the contraction device 1 is positioned as shown in FIG. 7, results in the septum and apex of the left ventricle being brought together for example with a combined force of about 8 Newtons, and a reduction in length of the linear actuator by about 14 mm. This has the effect of mechanically assisting the heart during systole and increasing the volume of blood pushed out of the left ventricle by about 15%.

When the system of the invention is to be used with the stomach, one or more contraction devices 1 may be implanted along any of the three main regions of the stomach 40, specifically the fundus 41, the body 43 and the pylorus 42, to move the walls of the stomach towards and away from each other. The contraction devices are implanted to generate optimal assistance during gastric emptying, which involves expelling chyme from the stomach 40 to the duodenum 44 through the pyloric sphincter (not shown). Once the contraction device is inserted in the stomach 40, it is anchored to the stomach wall through the tissue piercing anchoring assembly.

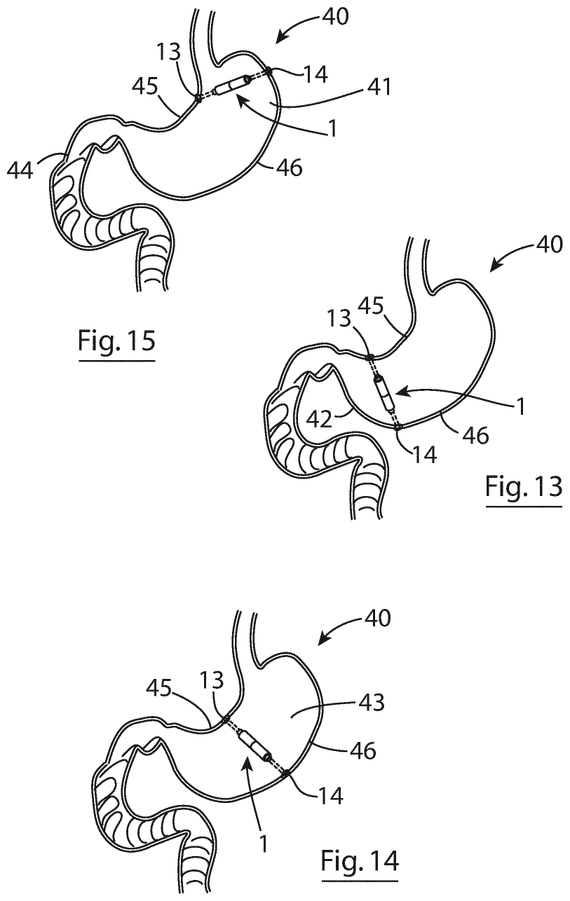

FIGS. 13 to 15 shows three exemplary configurations of the contraction device of the ICA system deployed in the stomach 40 to support the endogenous contractions of the stomach occurring during gastric emptying.

In patients with an impairment in the pylorus, the contraction device can be implanted in the pylorus 42 (pyloric antrum), as shown in FIG. 13 by means of the distal anchor 13 anchoring the device 1 to the lesser curvature 45 of the stomach 40 and the proximal anchor 14 anchoring the device 1 to the greater curvature 46 of the stomach 40. The pylorus is an advantageous implantation position, as pyloric antrum contraction in coordination with opening of the pyloric sphincter facilitates expulsion of chyme from the stomach.

FIG. 14 shows another configuration where the contraction device 1 is implanted in the body (mid antrum) 43 of the stomach 40 by means of the distal anchor 13 anchored to the lesser curvature 45 of the stomach 40 and the proximal anchor 14 anchored to the greater curvature 46 of the stomach 40.

FIG. 15 shows yet another configuration where the contraction device 1 is implanted in the upper fundus 41 of the stomach 40 by means of the distal anchor 13 anchored to the lesser curvature 45 of the stomach 40 and the proximal anchor 14 anchored to the greater curvature 46 of the stomach 40.

The contraction device may be deployed in the stomach through transesophageal endoscopy in a minimally invasive manner. In one embodiment, the contraction device may be delivered to the stomach by means of a 34 Fr delivery catheter. When the contraction device is designed for implantation in the stomach, the contraction device typically has a diameter of 11-14 mm, a retracted length of 50-55 mm, and an expanded length of about 80-100 mm.

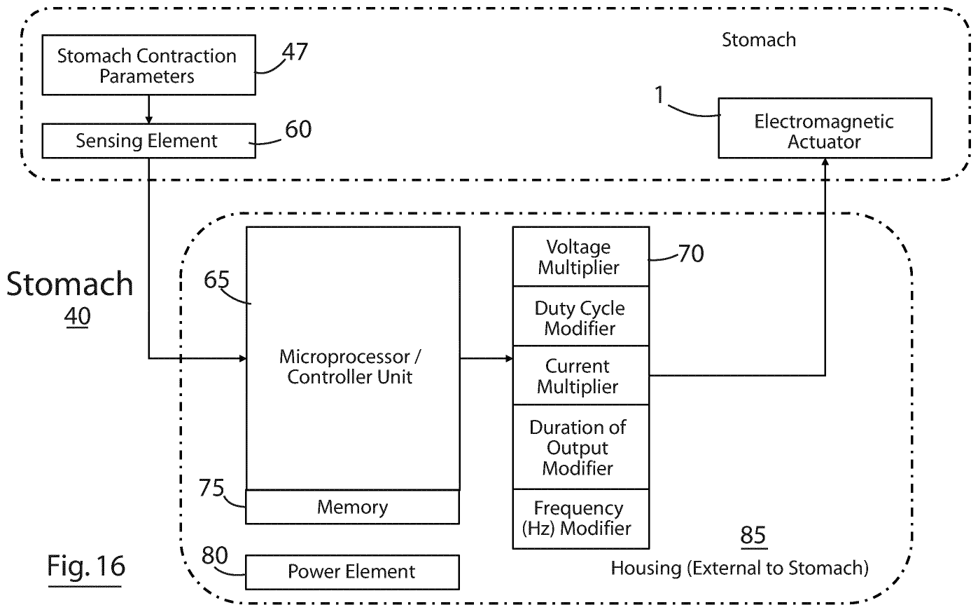

FIG. 16 shows a block diagram of one embodiment of the main components of the system of the invention when deployed for use in the stomach. In the embodiment shown in this figure, both the contraction device 1 in the form of an electromagnetic actuator and the sensor 60 are located in the stomach 40. The sensor 60 may be located on the stomach wall or on the anchor assembly. The sensor 60 is configured to detect stomach contraction parameters 90. In one embodiment the sensor comprises a transducer. A sensor may alternatively be provided external to stomach, such as shown in FIG. 17, where the pressure sensor and the power unit are located outside the stomach.

As per the block diagram of FIG. 10 in respect of the heart, the housing 85 which houses the controller 65, the electronic circuitry 70 for modifying the output parameters of the electromagnetic actuator, the memory 75 and the power unit 80 is located in the body outside the stomach.

The controller 65 is configured to adjust the output parameters of the contraction device 1 based on the received sensor data to provide a contraction sequence in coordination with antral contraction and pyloric sphincter opening. Accordingly, in use, the sensor 60 receives stimulus from the stomach wall. This stimulus may comprise pressure or electrical signals that travel through stomach muscles to control gastric contractions. The sensor 60 transmits the sensor data to the controller 65. The received data is analysed by the controller 65 according to the instructions stored in the memory 75. The controller 65 then delivers control signals to the power unit 80 and the electronic circuitry 70 so as to modify the output parameters of the contraction device 1 as appropriate based on the sensor data so as to activate the contraction device 1 to provide a contraction sequence in coordination with the natural contraction sequence of the stomach. The controller may be configured to deactivate the contraction device after predetermined criteria has been satisfied. For example, the controller may be configured to deactivate once the sensor 60 detects that the volume of chyme is reduced below a threshold.

When two contraction devices are implanted in the stomach, the contraction devices may be configured to pull the gastric walls of the stomach towards and away from each other repeatedly at specific travel distance, speed and force parameters to simulate the Migrating Myoelectric Complex (MMC) contraction sequence, whose function is to generate a peristaltic wave that propels chyme in an anterograde direction out of the GI tract. For example, a first contraction device can be deployed at a proximal segment of the stomach and a second contraction device can be deployed at a distal segment of the stomach. The first contraction device can be deployed to propagate at a first velocity of contraction (for example <1 cm/sec) while the second contraction device can be deployed to propagate at a second velocity which is faster than the first velocity (for example 3-4 cm/sec).

When the system of the invention is to be used with the bladder, the device may be implanted in the bladder to generate a contractile force on any wall of the bladder, or combination of the walls of the bladder, including the superior surface and the two infero-lateral surfaces of the bladder.

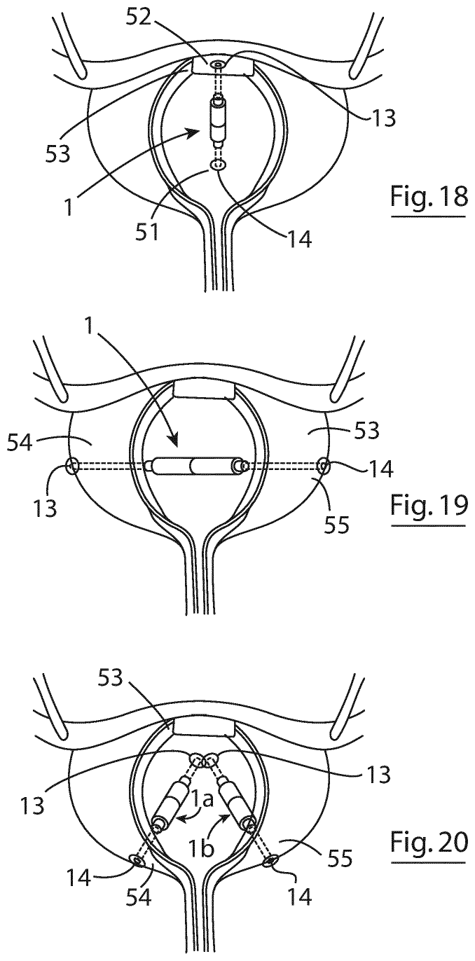

FIGS. 18 to 20 show three exemplary configurations of the contraction device of the ICA system deployed in the bladder to support the emptying of the bladder during urination (micturition). In one embodiment, the linear actuator has an expanded intraluminal (intra-bladder) length of 50-250 mm, 60-200 mm, and preferably about 120 mm.

FIG. 18 shows one exemplary configuration of intra-bladder placement of the contraction device, where the proximal anchor 14 of the contraction device 1 is adapted to engage the base (fundus) 51 of the bladder 50 and the distal anchor 13 of the contraction device 1 is adapted to engage with the apex 52 and the superior surface 53 of the bladder 50. In this position, the device functions to pull the apex 52 towards and away from the base 51.

The contraction device may also be positioned between the two infero-lateral surfaces of the bladder to pull the two infero-lateral walls of the bladder towards and away from each other. FIG. 19 shows an example of such an intra-bladder placement of the device, wherein the distal anchor 13 is adapted to engage with a first infero-lateral wall 54 of the bladder 50 and the proximal anchor 14 is adapted to engage with a second infero-lateral wall 55 of the bladder 50.

The device may alternatively be placed between the infero-lateral and the superior surface of the bladder to move the superior surface and infero-lateral surface towards and away from each other. FIG. 20 shows an example of such an intra-bladder placement, which comprises two contraction devices, 1a and 1b, wherein the proximal anchor 14 of the first device 1a is adapted to engage with the first infero-lateral wall 54 of the bladder 50 and the proximal anchor 14 of the second device 1b is adapted to engage with the second infero-lateral wall 55. The distal anchor 13 of the first device 1a and the distal anchor 13 of the second device 1b are then both adapted to engage with the superior surface 53 of the bladder 50.

In one embodiment, during the non-contracting phase of the contraction device, the intraluminal (intra-bladder) linear actuator exerts a pulling force that is close to zero Newtons, thereby minimizing any hindrance of the ability of the bladder to distend during the urine-filling phase.

In intra-bladder placement, the contraction device may be deployed into the bladder through the transurethral route in a minimally invasive manner by means of a transurethral catheter. In some embodiments, the contraction device and anchoring assembly may be delivered to the bladder in two steps, the first step comprising the anchoring assembly, and the second step comprising the contraction device. For example, anchors could be delivered to external walls of the bladder via a minimally invasive surgical procedure, or delivered percutaneously, and then the contraction device in the form of the linear actuator could be delivered separately to the target location via the urethra. The system could then be assembled in-situ by connecting the anchors (external to the bladder) to the linear actuator (internal to the bladder). In this embodiment, the coupling between at least one of the anchors and the contraction device comprises a magnet or magnetisable element. However, the electromagnetic coupling can also be solely used for delivery purposes.

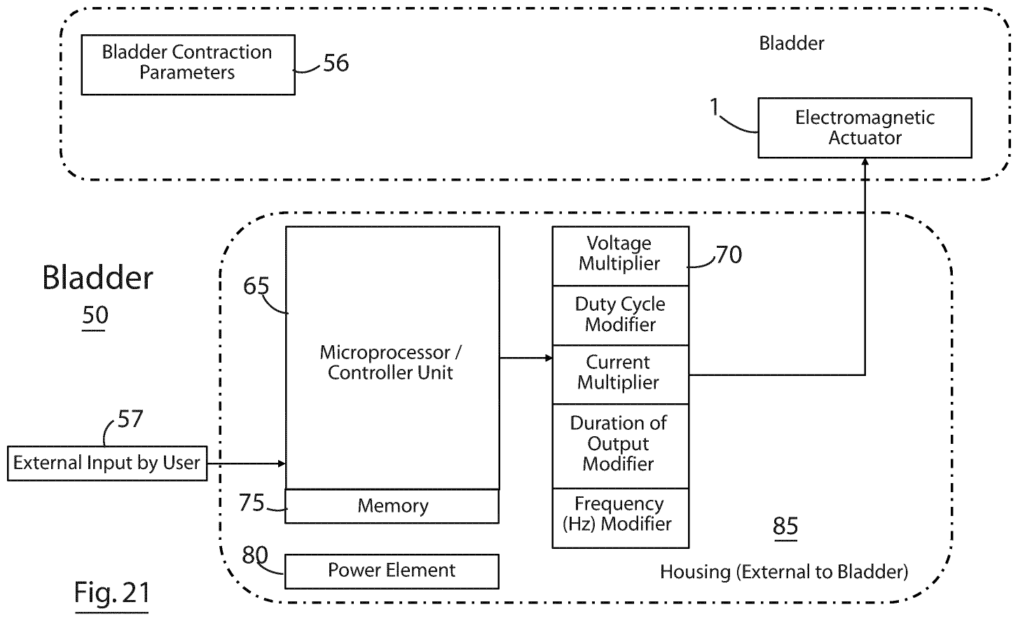

FIG. 21 shows a block diagram of one embodiment of the main components of the system of the invention when deployed for use in the bladder. In the embodiment shown in this figure, the contraction device 1 in the form of an electromagnetic actuator is located in the bladder 50. It should be noted that in this particular embodiment the system does not include a sensor, and the contraction device is user activated. User activation can be provided by means of a user activating an external control unit 57 which communicates with the controller 65, for example as shown in FIG. 22.

As per the block diagram of FIG. 10 in respect of the heart, the housing 85 which houses the controller 65, the electronic circuitry 70 for modifying the output parameters of the electromagnetic actuator, the memory 75 and the power unit 80 is located in the body outside the bladder. In another embodiment, the power unit is located external to the body.

In use, the user receives sensory input from the nervous system and naturally senses when the bladder is full. When appropriate, the user commences the urination process by naturally relaxing the external sphincter muscle. Concurrently the user activates the contraction device via the external control unit 57. This may be achieved for example by the user pressing a designated bar on the external control unit 57. A resulting signal is transmitted through conductive wire or through a wireless connection to the controller 65. The controller 65 then delivers control signals to the power unit 80 and the electronic circuitry 70 so as to modify the output parameters of the contraction device 1 as appropriate to contract and assist in ejection of urine from the body.

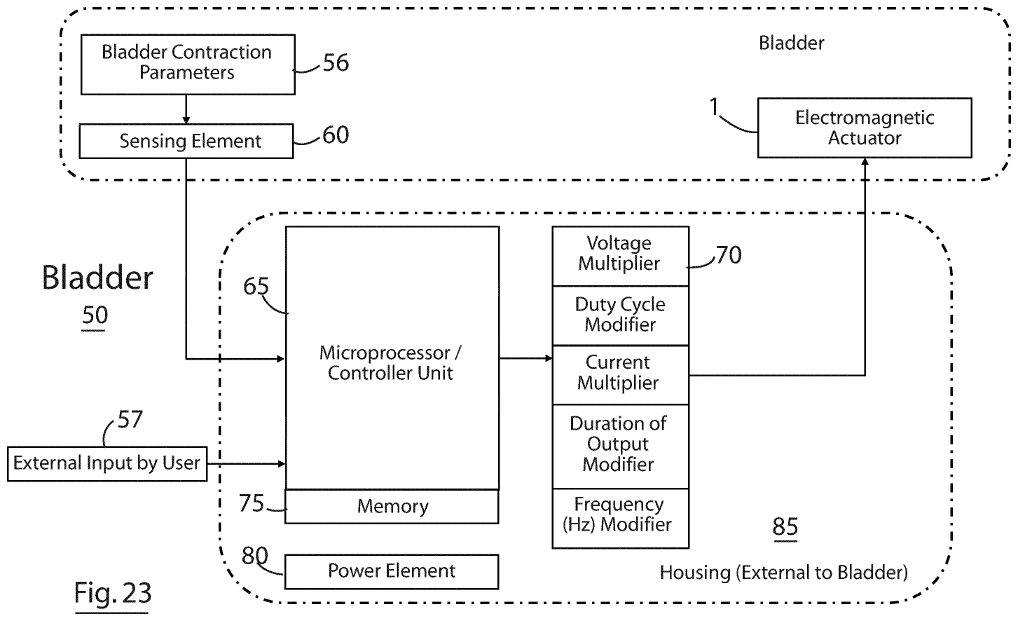

An alternative embodiment of the system of the invention for use with the bladder is shown in FIG. 23. In this embodiment, the system is configured to accommodate a patient who is unable to sense a full bladder. The system is identical to that shown in FIG. 21 except that it also includes a sensor for detecting when the bladder is full. Once the sensor detects a full bladder, it alerts the user (not shown in this figure). The user can then activate the contraction device manually via the external control unit 57 in communication with the controller 65. The controller 65 delivers control signals to the power unit 80 and to the electronic circuitry 70 so as to modify the output parameters of the contraction device 1 as appropriate to contract and assist in ejection of urine from the body. The controller can then deactivate the linear actuation functions of the contraction device 1 once the sensor 60 detects that the intra-bladder pressure has dropped below a predetermined threshold value.

### Supporting Data

A normal ejection fraction (EF) of the left ventricle is greater than 55%. This means that 55% of the total blood in the left ventricle is pumped out with each heartbeat. When the ejection fraction is 40% or less, heart failure with reduced ejection fraction occurs. This is when the cardiac muscle of the left ventricle is not pumping as well as normal.

Table 1 below summarizes the results of bench top testing validating the effects of the organ contraction device when applied to a simulated left ventricular vessel with reduced ejection fraction. The data indicates that when the organ contraction device is applied to a replicated left ventricular vessel with a reduced ejection fraction of 20 to 35%, the device can substantially improve the ejection fraction up to 2.2 litre/minute, depending on the force applied. This is a clinically significant improvement balancing the EF to borderline levels.

TABLE 1

| | 35% EF | | | | 30% EF | | | |
|---|---|---|---|---|---|---|---|---|
| | | Volume | | | | Volume | | |
| | Volume | per 70 | 35% | % | Volume | per 70 | 30% | % |
| Force | Collected | BPM | EF w/ | Increase | Collected | BPM | EF w/ | Increase |
| (N) | (ml) | (Litr) | SMAC | in EF | (ml) | (Litr) | SMAC | in EF |
| 0.0 | 27.0 | 1.9 | 33.8% | 0.0% | 21 | 1.47 | 28% | 0% |
| 2.0 | 29.5 | 2.1 | 36.9% | 9.3% | 24 | 1.68 | 32% | 14% |
| 3.0 | 30.0 | 2.1 | 37.5% | 11.1% | 25 | 1.75 | 34% | 19% |
| 4.0 | 32.0 | 2.2 | 40.0% | 18.5% | 27 | 1.89 | 36% | 29% |
| | 25% EF | | | | 20% EF | | | |
| | | Volume | | | | Volume | | |
| | Volume | per 70 | 25% | % | Volume | per 70 | 20% | % |
| Force | Collected | BPM | EF w/ | Increase | Collected | BPM | EF w/ | Increase |
| (N) | (ml) | (Litr) | SMAC | in EF | (ml) | (Litr) | SMAC | in EF |
| 0.0 | 15 | 1.05 | 22% | 0% | 13 | 0.91 | 20.0% | 0.0% |
| 2.0 | 17 | 1.19 | 24% | 13% | 14.5 | 1.015 | 22.3% | 11.5% |
| 3.0 | 20 | 1.40 | 29% | 33% | 15.5 | 1.085 | 23.8% | 19.2% |
| 4.0 | 22 | 1.54 | 32% | 47% | 16 | 1.12 | 27.0% | 35% |

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A system for augmenting the contraction of a contractile organ in a subject, comprising:
at least one implantable organ contraction device comprising an electronic linear actuation device for producing a contraction force;
an anchoring assembly for operably coupling the electronic linear actuation device to at least one wall of the contractile organ; and
a controller configured to modify the output parameters of the electronic linear actuation device so as to activate the electronic linear actuation device in a pattern synergistic to the natural contraction cycle of the contractile organ.

2. The system of claim 1, further comprising electronic circuitry for setting the output parameters of the electronic linear actuation device, wherein the electronic circuitry is coupled between the controller and the electronic linear actuation device, and a power unit associated with the electronic circuitry, wherein the controller is configured to send control signals to the electronic circuitry to modify the output parameters of the electronic linear actuation device.

3. The system of claim 2, wherein the electronic circuitry comprises one or more of: a voltage multiplier, a duty cycle modifier, a current multiplier, a duration of output modifier and a frequency modifier.

4. The system of claim 1, further comprising:
at least one sensor in communication with the controller for detecting one or more parameters associated with the contractile organ;
wherein the controller is configured to modify the output parameters of the electronic linear actuation device based on the one or more detected parameters.

5. The system of claim 4, wherein the contractile organ comprises the heart and wherein the at least one sensor comprises one of: a ventricle pressure sensor, a wireless pressure ventricular pressure sensor, a mems pressure sensor, an artery pressure sensor, a cardiac output (CO) sensor, a blood pressure sensor, a heart rate sensor, a motion sensor, an accelerometer, an
ECG (Electrocardiogram) sensor, an O2 saturation sensor, a microaccelerometer, a sonomicrometer, and a real-time contractility sensor.

6. The system of claim 4, wherein the anchoring assembly comprises a first anchor adapted to engage a septum of the heart ventricle and a second anchor adapted to engage a free wall of the heart ventricle, and wherein the electronic linear actuation device is configured to activate to extend and contract pulling the septum and free wall of the heart ventricle towards and away from each other repeatedly.

7. The system of claim 4, wherein the contractile organ comprises the stomach and wherein the at least one sensor comprises one of: a sensor for detecting electrical activity of the stomach, a sensor for detecting muscle contractility and a sensor for detecting chyme in the stomach beyond a specific volume.

8. The system of claim 7, wherein the controller is configured to deactivate the electronic linear actuation device upon the at least one sensor detecting the volume of chyme in the stomach to be below a predetermined threshold.

9. The system of claim 7, wherein the anchoring assembly comprises a first anchor adapted to engage one wall of the stomach and a second anchor adapted to engage another wall of the stomach, and wherein the electronic linear actuation device is configured to activate to expand and contract to move the walls of the stomach towards and away from each other.

10. The system of claim 1, wherein the contractile organ comprises the urinary bladder and wherein the system further comprises:
a user activatable external control unit in communication with the controller; wherein the controller is configured to modify the output parameters of the electronic linear actuation device upon activation of the external control unit by the subject.

11. The system of claim 10, further comprising:
at least one sensor in communication with the controller for detecting one or more parameters associated with the bladder;
wherein the at least one sensor is configured to alert the subject to activate the external control unit upon detection of a full bladder.

12. The system of claim 11, wherein the controller is configured to deactivate the electronic linear actuation device upon the at least one sensor detecting that the intra-bladder pressure has dropped below a predetermined threshold value.

13. The system of claim 11, wherein the at least one sensor comprises a sensor for measuring intravesical bladder pressure.

14. The system of claim 10, wherein the anchoring assembly comprises a first anchor adapted to engage one wall of the bladder and a second anchor adapted to engage another wall of the bladder, and wherein the electronic linear actuation device is configured to activate to expand and contract to move the walls of the bladder towards and away from each other.

15. The system of claim 1, wherein the electronic linear actuation device comprises at least one electromagnetic linear actuation device, and optionally wherein the at least one electromagnetic linear actuation device is configured to effect rotational actuation.
